(19) United States
(12) Patent Application Publication
(10) Pub. No.: US 2007/0178138 A1
(43) Pub. Date: Aug. 2, 2007

(54) Biodegradable non-opthalmic implants and related methods

(75) Inventors: Steven Pal, Coto De Caza, CA (US); Mitchell Brin, Newport Beach, CA (US)

Correspondence Address: Stephen Donovan;Allergan, Inc., 2525 Dupont Drive, Irvine, CA, 92612, US

(73) Assignee: ALLERGAN, INC., Irvine, CA (US)

(21) Appl. No.: 11/345,178

(22) Filed: Feb. 1, 2006

(51) Int. Cl. A61F 2/02; A61M 31/00

(52) U.S. Cl. 424/426; 604/500

(57) ABSTRACT

Biodegradable non-ophthalmic implants include one or more agents dispersed within a biodegradable polymer component. The implants release the agents from the biodegradable polymer component to a target site of a patient as the implant degrades. The agents provided in the implants can be therapeutic agents or diagnostic agents and are useful in medical treatments of non-ocular regions of a patient.

### BACKGROUND

The present invention relates to biodegradable non-ophthalmic implants and methods of making biodegradable implants and uses of biodegradable implants in medical therapy. The present implants include an agent that is released from the implant to a target site of a patient as the implant degrades in the patient's body.

Biodegradable polymers have been disclosed as being useful in forming drug delivery systems which can deliver drugs for extended periods of times. Such drug delivery systems have been disclosed as being provided in a variety of forms including compressed elements, injection molded elements, and extruded elements. In addition, a variety of different polymers have been disclosed as being useful in such drug delivery systems. For example, the following documents disclose various features of biodegradable drug delivery systems: U.S. Pat. No. 3,773,919; U.S. Pat. No. 4,767,628; U.S. Pat. No. 5,164,188; U.S. Pat. No. 5,443,505; U.S. Pat. No. 5,632,984; U.S. Pat. No. 5,766,242; U.S. Pat. No. 5,824,072; U.S. Pat. No. 5,869,079; U.S. Pat. No. 5,980,945; U.S. Pat. No. 5,980,948; U.S. Pat. No. 6,001,386; U.S. Pat. No. 6,007,843; U.S. Pat. No. 6,011,011; U.S. Pat. No. 6,312,708; U.S. Pat. No. 6,692,759; U.S. Pat. No. 6,383,509; and US 20040247645. In addition, numerous journal articles discuss polymeric drug delivery systems.

Although publications include descriptions of a variety of different polymers, a variety of different agents, and a variety of different elements to provide drug delivery to subjects, variations in polymer properties, agent properties, and implant type can influence the usefulness of implants in clinical practice.

Thus, there remains a need for new drug delivery systems and methods that can deliver medically useful agents to a patient and which are suitable for clinical practice. In other words, a problem apparent in the prior art is providing a biodegradable polymeric delivery system that can successfully deliver medically useful agents and successfully treat patients in need of treatment.

### SUMMARY

Biodegradable non-ophthalmic implants include or comprise a biodegradable polymer component and one or more medically useful agents. The present implants provide a prolonged or extended release of the medically useful agents to a target site in a patient, such as a human or non-human animal patient. Medically useful agents of the present implants include therapeutic agents and diagnostic agents, among others. Therapeutic agents can be understood to be agents, such as small molecule chemical compounds, nucleic acids, peptides, proteins, antibodies, and the like that are biologically active or that otherwise provide a therapeutic effect to a patient in need of medical treatment. Diagnostic agents can be understood to be agents which do not necessarily provide a direct therapeutic effect (e.g., the agents may not be therapeutically active or biologically active). Diagnostic agents include agents that may assist a physician in performing a medical treatment. For example, diagnostic agents may include one or more chemical agents useful in visualizing a target site within a patient, such as a target organ in need of treatment, or a patient's vasculature, and the like.

The present implants include a multi-extruded body member. The body member is in the form of a non-ophthalmic implant or implant element. For example, the present implants are structured, such as sized, shaped, or otherwise configured, to be implanted into a patient at a location other than an eye of the patient. In comparison to ophthalmic implants, non-ophthalmic implants can be larger, non-optically clear, and more rigid or more flexible since the target site for the present implants is located outside of the eye.

Embodiments of the present implants can be understood from the following description and claims.

In one embodiment, a biodegradable non-ophthalmic implant comprises, consists essentially of, or consists entirely of, a multi-extruded body member. For example, the implant may comprise, consist essentially of, or consist entirely of a double or triple extruded body member. Or, stated differently, the body member is an element formed by more than one extrusion process. The multi-extruded body member comprises, consists essentially of, or consists entirely of a poly (lactide-co-glycolide) (PLGA) copolymer and one or more medically useful agents. The medically useful agent is distributed throughout the PLGA copolymer in the form of a non-ophthalmic implant, as discussed herein. The medically useful agent is releasable from the implant to a non-ophthalmic target site. The PLGA copolymer of this embodiment comprises, consists essentially of, or consists entirely of about 75% by weight acid end PLGA and about 25% by weight ester end PLGA (3:1 acid to ester terminal end group ratio), and about 50% lactide and about 50% glycolide (1:1 lactide to glycolide ratio).

When the medically useful agent of the present implants is a therapeutic agent, the therapeutic agent is releasable from the implant to provide a desired therapeutic effect at a non-ophthalmic target site. The therapeutic effect persists for extended periods of time. For example, the therapeutic effect can persist for a time greater than the time required for the amount of the therapeutic agent to decrease below detectable levels at the target site.

The present implants can be understood to be neural implants, such as intrathecal implants, intracranial implants (implants structured for placement in a region of the brain), or intraspinal implants, dermal implants or intradermal implants, intraperitoneal implants, cardiac implants, joint implants, pancreatic implants, kidney implants, liver implants, prostate implants, and breast implants (including breast duct tissue implants).

Another embodiment of the present invention relates to methods of treating patients. For example, as described herein, a method of treating a patient comprises administering one or more of the present implants to a target site of a patient to treat, such as to reduce or alleviate, one or more symptoms of a condition experienced by a patient. Administration of the present implants which include therapeutic agents provides reduced side effects, enhanced dosing precision, or reduced frequency of administration relative to other systemic administration of identical therapeutic agents.

The present invention also encompasses the use of the present implants in treating a patient, such as in treating one or more of the conditions or diseases set forth herein, as well as medicaments, which are biodegradable non-ophthalmic implants, for treating such conditions or diseases by administering the implant to a target site of the patient. The invention also encompasses the use of a medically useful agent and a PLGA copolymer, as described herein, in the manufacture of a medicament for treating a patient.

As can be appreciated from the following description, each and every feature described herein, and each and every combination of two or more of such features, is included within the scope of the present invention provided that the features included in such a combination are not mutually inconsistent. In addition, any feature or combination of features may be specifically excluded from any embodiment of the present invention.

Additional aspects and advantages of the present invention are set forth in the following description, drawings and claims, particularly when considered in conjunction with the accompanying examples.

### BRIEF DESCRIPTION OF THE DRAWINGS

FIG. 1 is a perspective view of a biodegradable non-ophthalmic implant comprising a medically useful agent.

FIG. 2 is an illustration of the implant of FIG. 1 in an implant injecting device.

### DESCRIPTION

Biodegradable non-ophthalmic implants include or comprise a biodegradable polymer component and one or more medically useful agents. The present implants provide a prolonged or extended release of the medically useful agents to a target site in a patient, such as a human or non-human animal patient. Medically useful agents of the present implants include therapeutic agents and diagnostic agents, among others. Therapeutic agents can be understood to be agents, such as small molecule chemical compounds, nucleic acids, peptides, proteins, antibodies, and the like that are biologically active or that otherwise provide a therapeutic effect to a patient in need of medical treatment. Diagnostic agents can be understood to be agents which do not necessarily provide a direct therapeutic effect (e.g., the agents may not be therapeutically active or biologically active). Diagnostic agents include agents that may assist a physician in performing a medical treatment. For example, diagnostic agents may include one or more chemical agents useful in visualizing a target site within a patient, such as a target organ in need of treatment, or a patient's vasculature, and the like.

The present non-ophthalmic implants deliver one or more agents to focal areas or target sites of disease or injury that are located outside of the eye of a patient. As discussed herein, the agents can be therapeutic agents or diagnostic agents. As used herein, a target site can be understood to refer to a region of a patient's body adjacent or near an implanted implant. The target site may surround the implant when the implant is placed completely in a body structure, such as an organ or other tissue. Or the target site may be adjacent the implanted implant when the implant is placed next to a body structure. In certain situations, the target site refers to a body region of a patient, such as an injured or diseased body region, located within five inches of the implant. In more specific situations, the target site may be a body region located within about three inches of the implant. As used herein, the term about can be understood to refer to plus or minus 10% of a given value. It can be understood, therefore, that the present implants can provide controlled, precise, steady doses of agents to non-ocular organs or other body tissues or structures to achieve a desired effect. Thus, with the present implants, localized focal delivery of medically useful agents can be obtained to assist a physician in treating a patient.

When the implants include one or more therapeutic agents, the present delivery of the therapeutic agent is associated with reduced side effects, enhanced drug dosing precision, reduced dosing frequency, and reduced surgical risk and intervention relative to existing medical therapies, such as systemic administration of therapeutic agents.

The present implants can be administered to a patient to a variety of conditions or diseases of a patient. For example, certain implants can be administered to treat cancer of a patient, such as by reducing tumor growth or tumor size. Certain implants can be administered to treat arthritis, or joint injuries or conditions. Certain implants can be administered to treat lesions, such as a skin lesion, vascular lesion, neoplastic lesion, or an infectious lesion. Certain implants can be administered to treat cardiac conditions, and to generally improve cardiac and/or vascular function. Certain implants can be administered to spinal regions to provide long acting pain relief and/or spasticity relief. Certain implants can be administered to the central nervous system to provide neuroprotective effects of neurons of the central nervous system, and thereby provide enhanced function of brain neuronal activity. In addition, certain implants can be administered to the central nervous system to treat central nervous system tumors. For example, certain implants can be implanted within one or more ventricles of the central nervous system to treat a central nervous system disorder, disease, or condition. With the present implants, chronic conditions can be successfully treated. The success of treating such chronic conditions can be related to the prolonged delivery of the therapeutic agents from the implants, the precise dosing achieved by the release rate of the implants and the reduced side effects so associated, and the increased patient compliance since the frequency of administration of the therapeutic agents can be reduced.

Reference will now be made in detail to current embodiments of the present invention. Although the disclosure herein refers to certain embodiments, it is to be understood that these embodiments are presented by way of example and not by way of limitation. The intent of the following detailed description, although discussing exemplary embodiments, is to be construed to cover all modifications, alternatives, and equivalents of the embodiments as may fall within the spirit and scope of the invention as defined by the appended additional disclosure.

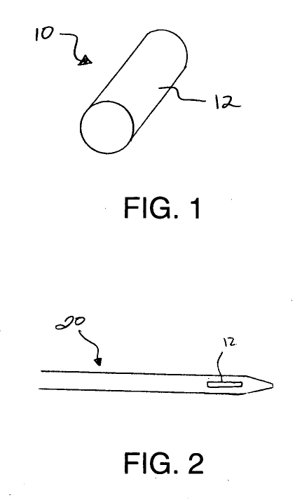

A biodegradable non-ophthalmic implant in accordance with the present disclosure comprises, consists essentially of, or consists entirely of a multi-extruded body member. For example, as shown in FIG. 1, a biodegradable non-ophthalmic implant 10 has a multi-extruded body member 12. The body member is a structure or element formed by more than one extrusion process. In certain embodiments, the body member is a double extruded element. In other embodiments, the body member is a triple extruded element. In further embodiments, the body member may have been formed from more than three different extrusion steps. Multi-extruded non-ophthalmic implants in accordance with the present disclosure appear to provide enhanced delivery of medically useful agents and enhanced treatment of patients suffering from disease or injury, and the like.

The multi-extruded body member comprises, consists essentially of, or consists entirely of a poly (lactide-co-glycolide) (PLGA) copolymer and one or more medically useful agents. The medically useful agent or agents is distributed throughout the PLGA copolymer in the form of a non-ophthalmic implant. For example, the combination of the PLGA copolymer and the medically useful agent(s) are in the form of an implant that is structured, such as sized and/or shaped, to be implanted in a location outside of the eye of a patient. For example, when implanted in a patient, the present implants do not contact a portion of an eye, such as the interior of the eye or the exterior of the eye, or related ocular structures.

The medically useful agent(s) is releasable from the implant, or body member, to a non-ophthalmic target site of a patient. Thus, the present implants are structured or otherwise configured to release the medically useful agent to one or more regions other than an ocular region. The release of the agent(s) is controlled based on the properties of the biodegradable polymer component of the present implants. For example, the release of the agent(s) can be related to the particular ratio of end groups of the polymers, such as terminal acid end groups and/or terminal ester end groups, and/or the ratio of lactide to glycolide present in the copolymer. As discussed herein, the present embodiment of the non-ophthalmic implants comprise specific types of PLGA copolymers. The specific PLGA copolymers described herein can provide the benefits obtained by the present implants, such as reduced side effects, enhanced drug dosing precision, and reduced frequency of dosing. These benefits can be substantial since, unlike ocular and intraocular implants, the present implants are administered to regions that can be more intimate with the circulatory system of the patient.

The PLGA copolymer of the present implants comprises, consists essentially of, or consists entirely of, a PLGA copolymer comprising about 75% by weight acid end PLGA and about 25% by weight ester end PLGA, and about 50% lactide polymer and about 50% glycolide polymer. Such terminology has its art-recognized meanings.

In other implants, the biodegradable polymer component comprises one or more biodegradable polymers that have stabilities, degradation rates, melt temperatures, melt flow indices, and other properties, similar to the PLGA copolymer described above. Such biodegradable polymers can be identified using routine methods known to persons of ordinary skill in the art and methods disclosed herein.

As described herein, embodiments of the foregoing implants may comprise a body member that is a double extruded body member of the PLGA copolymer and the medically useful agent.

The medically useful agent can be a therapeutic agent and/or a diagnostic agent. Examples of therapeutic agents and diagnostic agents are described in more detail herein. The present invention encompasses implants which specifically include one of each of the agents, and combinations of two or more of the agents, described herein. When an implant comprises a therapeutic agent, the therapeutic agent can be homogenously distributed in a PLGA copolymer matrix. The therapeutic agent is releasable from the implant and provides a therapeutic effect at a non-ophthalmic target site. The therapeutic effect can persist for a time beyond which the therapeutic agent is detectable at the target site. In other words, the implant has an extended therapeutic effect. For example, the therapeutic effect persists longer than the therapeutic level of the agent is detectable at the target site. For example, the therapeutic effect may persist for a time beyond which 95% of the therapeutic agent has been released, or for which 80% of the therapeutic agent has been released.

Certain of the present implants comprise a medically useful agent that is selected from the group consisting of chemotherapeutic agents, anti-inflammatory agents, analgesic agents, anti-spasmodic agents, neuroprotective agents, and combinations thereof.

Chemotherapeutic agents refer to agents that are useful in treating cancer. Chemotherapeutic agents of the present implants include agents that reduce neovascularization, prevent further neovascularization from the time of administration of the implants, reduce tumor size, prevent further tumor growth from the time of administration of the implants, and/or destroy or inhibit cancerous cell growth or activity. Examples of chemotherapeutic agents that can be included in the present implants include, without limitation, all-trans retinoic acids, azacitidine, azathioprine, bleomycin, carboplatin, capecitabine, cisplatin, chlorambucil, cyclophosphamide, cytarabine, daunorubicin, docetaxel, doxifluridine, doxorubicin, epirubicin, etoposide, fluorouracil, gemcitabine, hydroxyurea, idarubicin, mechlorethamine, mercaptopurine, methotrexate, mitoxantrone, oxaliplatin, paclitaxel, pemetrexed, teniposide, thioguanine, valrubicin, vinblastine, vincristine, vindesine, and vinorelbine. Additional examples of antineoplastic agents include adriamycin, actinomycin, mitomycin, carmustine (BCNU), methyl-CCNU, interferons, phenesterine, taxol and derivatives thereof, taxotere and derivatives thereof, tamoxifen, piposulfan, and flutamide, and derivatives thereof.

As used herein, the term “derivative” refers to any substance which is sufficiently structurally similar to the material of which it is identified as a derivative so as to have substantially similar functionality or activity, for example, therapeutic effectiveness, as the material when the substance is used in place of the material. Useful derivatives of a substance can be routinely determined by conducting one or more conventional assays using the derivatives instead of the substance from which the derivative is derived.

Anti-inflammatory agents useful in certain of the present implants include without limitation both steroids and non-steroidal anti-inflammatory drugs, and combinations thereof. Some specific examples of steroids used in the present implants include corticosteroids, such as cortisone, prednisolone, flurometholone, dexamethasone, medrysone, loteprednol, fluazacort, hydrocortisone, prednisone, betamethasone, prednisone, methylprednisolone, triamcinolone hexacatonide, paramethasone acetate, diflorasone, fluocinonide, fluocinolone, triamcinolone, triamcinolone acetonide, derivatives thereof, and mixtures thereof. Examples of non-steroid antiinflammatory drugs include aspirin, ibuprofen, ketorlac tromethamine (Acular), and the like.

Neuroprotective agents useful in the present implants include agents that preserve cellular viability and/or activity. In certain implants, neuroprotective agents can be anti-excitotoxic agents, including without limitation, glutamate receptor antagonists or glutamate receptor blockers, such as NMDA receptor antagonists or blockers, and gamma-aminobutyric acid receptor or glycine receptor agonists, and the like. In other implants, neuroprotective agents can be alpha 2 adrenergic receptor agonists. In at least one embodiment, a biodegradable non-ophthalmic implant comprises, consists essentially of, or consists of an alpha 2 adrenergic agonist, such as brimonidine, a salt thereof, such as brimonidine tartrate, or combinations thereof, as the therapeutically active agent.

The present implants may also include one or more different therapeutic agents other than those described above. Therapeutic agents which may be provided in the present implants may be obtained from public sources or may be synthesized using routine chemical procedures known to persons of ordinary skill in the art. Agents are screened for therapeutic efficacy using conventional assays known to persons of ordinary skill in the art. For example, agents can be monitored for their effects on reducing inflammation, reducing pain, reducing spasticity, preventing tumor growth, reducing excitoxic cell death, and the like using such conventional assays.

Thus, the present implants may comprise one or more of the following: anti-histamine agents, antibiotic agents, beta blocker agents, immunosuppressive agents, anti-viral agents, anti-oxidant agents, adrenergic receptor agonists and antagonists, and VEGF inhibitor agents.

Examples of antihistamines include, and are not limited to, loradatine, hydroxyzine, diphenhydramine, chlorpheniramine, brompheniramine, cyproheptadine, terfenadine, clemastine, triprolidine, carbinoxamine, diphenylpyraline, phenindamine, azatadine, tripelennamine, dexchlorpheniramine, dexbrompheniramine, methdilazine, and trimprazine doxylamine, pheniramine, pyrilamine, chiorcyclizine, thonzylamine, and derivatives thereof.

Examples of antibiotics include without limitation, cefazolin, cephradine, cefaclor, cephapirin, ceftizoxime, cefoperazone, cefotetan, cefutoxime, cefotaxime, cefadroxil, ceftazidime, cephalexin, cephalothin, cefamandole, cefoxitin, cefonicid, ceforanide, ceftriaxone, cefadroxil, cephradine, cefuroxime, cyclosporine, ampicillin, amoxicillin, cyclacillin, ampicillin, penicillin G, penicillin V potassium, piperacillin, oxacillin, bacampicillin, cloxacillin, ticarcillin, azlocillin, carbenicillin, methicillin, nafcillin, erythromycin, tetracycline, doxycycline, minocycline, aztreonam, chloramphenicol, ciprofloxacin hydrochloride, clindamycin, metronidazole, gentamicin, lincomycin, tobramycin, vancomycin, polymyxin B sulfate, colistimethate, colistin, azithromycin, augmentin, sulfamethoxazole, trimethoprim, gatifloxacin, ofloxacin, and derivatives thereof.

Examples of beta blockers include acebutolol, atenolol, labetalol, metoprolol, propranolol, timolol, and derivatives thereof.

Examples of immunosuppresive agents include cyclosporine, azathioprine, tacrolimus, and derivatives thereof.

Examples of antiviral agents include interferon gamma, zidovudine, amantadine hydrochloride, ribavirin, acyclovir, valciclovir, dideoxycytidine, phosphonoformic acid, ganciclovir and derivatives thereof.

Examples of antioxidant agents include ascorbate, alpha-tocopherol, mannitol, reduced glutathione, various carotenoids, cysteine, uric acid, taurine, tyrosine, superoxide dismutase, lutein, zeaxanthin, cryotpxanthin, astazanthin, lycopene, N-acetyl-cysteine, carnosine, gamma-glutamylcysteine, quercitin, lactoferrin, dihydrolipoic acid, citrate, Ginkgo Biloba extract, tea catechins, bilberry extract, vitamins E or esters of vitamin E, retinyl palmitate, and derivatives thereof.

Some additional examples of therapeutic agents include anacortave (anti-angiogenesis compound), hyaluronic acid, ranibizumab, pegaptanib (Macugen) (VEGF inhibitors), cyclosporine, gatifloxacin, ofloxacin, epinastine (antibiotics). Other therapeutic agents include squalamine, carbonic anhydrase inhibitors, brimonidine, prostamides, prostaglandins, antiparasitics, antifungals, tyrosine kinase inhibitors, and derivatives thereof.

The therapeutic agent is provided in the present implants in therapeutic amounts. Or, stated differently, the present implants comprise amounts of therapeutic agent or agents that can be released from the implant at a target site in therapeutically effective amounts. The amounts may be understood to be symptom relieving amounts, such as an amount effective in alleviating or reducing one or more symptoms of a disease or condition experienced by the patient. In certain implants, the amount of therapeutic agent is a neuroprotective amount. In other implants, the amount of the therapeutic agent is an anti-inflammatory amount. In still further implants, the amount of the therapeutic agent is a tumor reducing amount. The present implants release such therapeutic amounts of the therapeutic agents at precise doses for extended periods of time. By maintaining a desired delivery dose of the therapeutic agent, it is possible to treat a disease or alleviate a symptom of a disease with reduced side effects compared to other administration techniques.

The therapeutic agent may be in a particulate or powder form and may be associated with the biodegradable polymer in a number of different configurations. For example, particles of the therapeutic agent may be entrapped by a polymer matrix, such as a biodegradable polymer matrix. Or, therapeutic agent particles may be encompassed by the polymeric component, such as in the form of a diffusion controlled implant. In certain embodiments, the therapeutic agent is homogenously distributed throughout the implant. For example, the concentration of the therapeutic agent does not vary by more than 20% for any two adjacent regions of the implant before administration to a patient. In some implants, the concentration variation may be less than 10%, or even less than 5% throughout the implant.

In certain embodiments, therapeutic agent particles in the present drug delivery systems may have an effective average size less than about 3000 nanometers. In other embodiments, the particles may have an effective average size greater than 3000 nanometers. In certain implants, the particles may have an effective average particle size about an order of magnitude smaller than 3000 nanometers. For example, the particles may have an effective average particle size of less than about 500 nanometers. In additional implants, the particles may have an effective average particle size of less than about 400 nanometers, and in still further embodiments, a size less than about 200 nanometers. The particles of the therapeutic agent may be associated with the polymer to form products suitable for extrusion, as discussed herein.

The therapeutic agent of the present implants may be present in an amount from about 1% to 90% by weight of the implant. More preferably, the therapeutic agent is present in an amount from about 20% to about 80% by weight of the implant. In a preferred embodiment, the therapeutic agent comprises about 40% by weight of the implant (e.g., 30%-50%). In another embodiment, the therapeutic agent comprises about 60% by weight of the implant.

The present implants are structured to be inserted or placed at a target site of a patient. For example, an implant may be placed in or near neural tissue, such as in the central nervous system. Such an implant has physical features, such as maximum dimensions, geometric configurations, and surface features, which contribute to the compatibility of the implant with neural tissue. Such an implant can be understood to be a neural implant. Neural implants include intrathecal implants, intracranial implants, intraventricular, and intraspinal implants. Such implants can be administered into an intrathecal target site, an intracranial target site, such as the brain, including a ventricle of the brain, or into an intraspinal target site, such as within or between one or more vertebrae of the patient.

Certain of the present implants are cardiac implants. For example, the implants are structured and configured to be placed in or adjacent cardiac tissue. Certain of the implants can be inserted into cardiac muscle, such as a portion of a heart of a patient. Other implants may be inserted into an artery or similar cardiac vessel. If the implant is being administered to treat a cardiac or vascular lesion, the implant can be placed locally into the lesion or adjacent to the vascular lesion.

Joint implants refer to implants that are structured and/or configured to be placed in or adjacent a joint of a patient. Certain implants can be placed between two adjacent bones and provide therapeutic benefits for prolonged periods of time without being compromised by movements of the bones. Certain of the implants can be secured to bone tissue, cartilage tissue, and the like. Certain implants are structured for placement in synovial fluid of the joint.

The present implants may include a body member, as described herein, in the form of pancreatic implants, intraperitoneal implants, dermal implants, kidney implants, liver implants, prostate implants, and breast implants. Such implants are structured and/or configured to be administered to their respective target organs. Breast implants include implants structured to be administered to breast duct tissue, as well as other breast tissue. For example, a pancreatic implant could be administered in or near a pancreas of a patient to treat a pancreatic condition. An intraperitoneal implant could be administered in the peritoneal cavity to treat an intraperitoneal condition or disease. Dermal implants, including subdermal and intradermal implants can be administered to treat skin conditions, including skin lesions and/or tumors or other forms of cancer.

In certain embodiments, the present implants are selected from the group consisting of joint implants, intraventricular implants, intraperitoneal cavity implants, intratumor implants, and intralesion implants. An intralesion implant is structured to be placed in a lesion of a patient, including a lesion selected from the group consisting of dermal lesions, vascular lesions, neoplastic lesions, and the like. In addition, implants can be structured for placement in proximity to a lesion or tumor without being placed in the lesion or tumor to release a therapeutic agent or agents to treat the lesion or tumor.

As can be appreciated from the present description, the therapeutic agent of the implant can be released from the implant and be associated with reduced side effects, enhanced dosing precision, and/or reduced frequency of administration relative to systemic administration of an identical therapeutic agent.

The present implants can be administered to a target site using any suitable technique. Preferably, the technique is minimally invasive to enhance recovery rate and overall patient comfort. In one embodiment, the implants can be inserted using an implant injecting device. In some embodiments, the implants are inserted through a trocar, which may be coupled to a syringe-like device or other piston driven apparatus. Certain implants may be surgically placed in or near a target site using an implant retention device, such as forceps and the like. Certain implant delivery devices are similar to the devices disclosed in U.S. Patent Publication No. 20050203542. However, the present devices are not used to administer the present implants into the eye or other ophthalmic tissue. Thus, the present invention also encompasses an implant injecting device comprising one or more of the implants described herein. Similarly, the present implants may be provided in an implant injecting device, such as shown in FIG. 2. Certain of the injecting devices may include a fiber optic component or element which may be helpful in visualizing the target site for the implant.

The present implants comprise a multi-extruded body member, as described herein. The implants are produced by combining a PLGA copolymer and one or more medically useful agents to form a mixture. The mixture can be extruded to form a first extruded product. The resulting first extruded product can be processed to produce material suitable for a second extrusion process. The material is extruded again to form an implant or an extruded filament or similar device. When the second extruded product is a filament, it may be further processed to produce one or more of the present implants suitable for placement in or near a non-ophthalmic target site of a patient in need of treatment.

The implants can be sterilized, such as by using gamma radiation, e-beam radiation, or other sterilization technique. The multi-extrusion of the materials with the particular polymers disclosed herein may be effective in providing the desired release properties and therapeutic benefits while withstanding perturbations of the implant due to sterilization, packaging, and other processing steps.

In addition, the present materials can be subjected to one or more additional extrusion steps to form implants with improved properties. Desirably, the extrusion and processing steps are optimized to maintain the activity of the medically useful agent and to prevent or reduce negative alterations in the release rates, stability, and other physical properties of the implants.

The produced implants can be in the form of rods, cylinders, rings, discs, ellipses, spheres, random particle shapes, cubes, and the like. Certain of the implants are sufficiently flexible to accommodate different target structures. Other implants are relatively rigid. Implants can be coated if desired with a polymeric material to further influence the release rates and stability of the implants.

The implants can have a maximum dimension, such as length or diameter from 0.5 mm to 20 mm. For example, the present implants can have a maximum length greater than about 1 mm, or the implants can have a maximum length less than about 15 mm.

The proportions of therapeutic agent, polymer, excipient agents, and any other modifiers may be empirically determined by formulating several drug delivery elements with varying proportions. In addition, other biodegradable polymers having similar properties to the PLGA copolymer disclosed herein can be identified using routine methods. For example, a USP approved method for dissolution or release test can be used to measure the rate of release (USP 23; NF 18 (1995) pp. 1790-1798). For example, using the infinite sink method, a weighed sample of the element is added to a measured volume of a solution containing 0.9% NaCl in water, where the solution volume will be such that the drug concentration after release is less than 5% of saturation. The mixture is maintained at 37° C. and stirred slowly to maintain the elements in suspension. The appearance of the dissolved drug as a function of time may be followed by various methods known in the art, such as spectrophotometrically, HPLC, mass spectroscopy, etc. until the absorbance becomes constant or until greater than 90% of the drug has been released.

The present implants can be administered to a patient, such as a human or non-human animal patient, to treat a condition or disease of the patient. As used herein, treatment refers to the reduction or alleviation of one or more symptoms associated with a disease or condition. The reduction can be subjective based on the patient's own perception, or the reduction can be objective as determined by the physician or quantified using a scale with values selected by the patient. Symptoms that can be alleviated include pain, discomfort, spasticity, tumor growth, inflammation, cognitive dysfunction, memory loss, stuttering, and the like.

Symptomatic relief can be observed with 1 week after the surgery, and such relief can increase and be maintained for several weeks and for at least 2 weeks or more without further administration of another implant. Certain implants can provide symptomatic relief for at least three months, such as for at least six months, or even for more than a year. Thus, the present implants provide relief of both acute and chronic symptoms of the patient. The relief provided by a single administration of the present implants is maintained for extended periods of time. In comparison, systemic administration of therapeutic agents in liquid compositions provide relief on the order of hours, and frequent dosing is often required to maintain a desired relief.

In certain methods, the implant is administered to a cancerous target site, such as to a tumor or near a tumor of the brain, skin, pancreas, kidneys, liver, prostate gland, and/or breasts. The implants can also be administered to such organs to treat pre-cancerous tissue and thereby prevent the formation of cancerous tumors.

In other methods, the implant is administered to a joint. For example, the implant can be administered to an arthritic joint or injured joint. The prolonged release of the therapeutic agent, and the prolonged relief provided thereby, can effectively relieve joint pain, joint inflammation, or treat joint disease.

Further methods may include administering the implant to a cardiac target site, such as a heart muscle or cardiac artery to enhance cardiac or vascular function in a patient in need of treatment or relief, or to treat a cardiac or vascular lesion or tumor.

Still further implants can be implanted to a central nervous system target site. For example, an intraspinal or intrathecal implant can be administered into or near the spine to release an analgesic agent or antispasmodic agent to provide long lasting pain or spasticity relief. Other implants can be intrathecally administered and release alpha 2 adrenergic receptor agonists, such as brimonidine, to enhance brain function by reducing neurodegeneration resulting from an injury or insult. Such implants may be particularly useful in preventing further brain dysfunction resulting from stroke, ischemia, or other damage to the brain.

The present invention also encompasses combination therapies. For example, a method may include administering one of the present implants which comprises a steroid to a central nervous system target site, and administering a second implant which comprises a chemotherapeutic agent into a different target site to treat a cancerous tumor of the different target site.

Embodiments of the present implants may comprise an excipient component or may be provided in compositions comprising an excipient component. Any conventional excipient agent which is useful in liquid compositions, such as formulations, suspensions, and the like, or is useful in polymeric devices may be used in the present implants. Examples of excipient agents include viscosing agents or viscosity inducing agents, solubilizing agents, preservative agents, buffer agents, or tensioactive agents.

Viscosing agents include, without limitation, sodium carboxymethylcellulose (CMC), hydroxypropylmethyl cellulose (HPMC), poloxamer 407nf (Pluronic® F127 Prill), and hyaluronic acid.

Solubilizing agents include without limitation, cyclodextrins (CDs), such as hydroxypropyl gamma-CD (Cavasol®), sulfobutyl ether 4 beta-CD (Captisol®), and hydroxypropyl beta-CD (Kleptose®).

Preservative agents may include benzyl alcohol.

Buffer agents may include phosphate buffers, such as dibasic sodium phosphate heptahydrate, monobasic sodium phosphate monohydrate; and/or borate buffers, such as sodium borate, boric acid, sodium chloride (according to Eu. Pharmacopeia).

Resuspension agents may include polysorbate 80 (Tween80®).

Tensioactive agents may include sodium chloride sugar alcohols, such as mannitol.

Additional aspects of the present invention are provided in the following non-limiting examples which are not intended to limit the scope of the invention.

### EXAMPLES

### Example 1

### Production of Biodegradable Non-ophthalmic Implants

A PLGA copolymer having about 75% by weight terminal acid groups and 25% by weight terminal ester groups (e.g., an acid end group to ester end group ration of 3:1) and having a lactide content of about 50% and a glycolide content of about 50% (e.g., a lactide to glycolide ration of 1:1) can be milled using a vibratory feeder and grinding nozzle to form particles of the copolymer. The particles can be sorted or formed to produce a population of particles having a pre-determined size, such as a diameter of about 20 μm.

Particles of one or more medically useful agents can be combined with the biodegradable polymer particles to form a blended mixture. The blended mixture can then be extruded using an extrusion device, such as a Haake Twin Screw Extruder, to form an extruded composition or product, such as an extruded filament. The extruded product can then be pelletized or otherwise processed to produce smaller products. The pelletized extruded product can then undergo a second extrusion step to produce a double-extruded element comprising a biodegradable polymer and at least one medically useful agent. The double extruded element can be in the form of a non-ophthalmic implant, or it can be in the form of a larger product, such as a filament, which can be processed to form implants as disclosed herein.

Implants can also be made as set forth in U.S. Patent Publication No. 20050048099.

In certain implants, the medically useful agent makes up about 50% by weight of the implant and the PLGA polymer component makes up about 50% by weight of the implant. In other implants, the medically useful agent can constitute up to about 80% by weight of the implant or can constitute down to about 20% by weight of the implant with the remaining weight percent comprising the PLGA copolymer.

Batches of implants can be produced using the foregoing process. Such batches can have average implant sizes or weights. For example, one batch may have an average implant weight of about 1 mg, one batch may have an average implant weight of about 3 mg, and one batch may have an average implant weight of about 5 mg.

The implants can be administered using an implant injecting device. In addition, the present implants may be provided in an injecting device and packaged for distribution to a medical facility or physician for single use administration.

### Example 2

### Breast Cancer Therapy

A 48 year old woman is diagnosed with breast cancer. The woman presents with a 2 cm malignant tumor in the upper quadrant of her left breast. Regional spread of the cancer is not apparent. An implant as described in Example 1 is administered into the tumor. The implant comprises a chemotherapeutic agent that is released from the implant for at least about 2 weeks. Observation of tumor regression is indicative that the treatment is successful. The patient is cured of the breast cancer without requiring a lumpectomy or similar surgical procedure.

### Example 2A

### Breast Cancer Therapy with Herceptin

The procedure as set forth in Example 2 can be repeated using a biodegradable non-ophthalmic implant that comprises effective amounts of Trastuzumab (Herceptin®; Genentech, CA).

### Example 2B

### Breast Cancer Therapy with Taxol

The procedure as set forth in Example 2 can be repeated using a biodegradable non-ophthalmic implant that comprises effective amounts of paclitaxel (Taxol®; Bristol-Myers Squibb Company, NY).

### Example 3

### Prostate Cancer Therapy

A 58 year old man is diagnosed with prostate cancer (stage T2c). The man has a tumor extending into both prostate gland lobes. Regional spread of the cancer is not apparent. An implant as described in Example 1 is administered into the tumor. The implant comprises a chemotherapeutic agent that is released from the implant for at least about 2 weeks. Observation of tumor regression and prostate serum antigen (PSA) reduction is indicative that the treatment is successful. The patient is cured of the prostate cancer without requiring surgical removal of the prostate.

### Example 3A

### Prostate Cancer Therapy with Mitoxantrone

The procedure as set forth in Example 3 can be repeated using a biodegradable non-ophthalmic implant that comprises effective amounts of mitoxantrone (Novantrone®; OSI Pharmaceuticals, NY).

### Example 3B

### Prostate Cancer Therapy with Vinblastine

The procedure as set forth in Example 3 can be repeated using a biodegradable non-ophthalmic implant that comprises effective amounts of vinblastine (Velban®; Eli Lilly, IN).

### Example 4

### Pancreatic Cancer Therapy

A 62 year old man is diagnosed with pancreatic cancer (T2 tumor size). The man has a tumor which is limited to the pancreas. Regional spread of the cancer is not apparent. An implant as described in Example 1 is administered into the tumor. The implant comprises the chemotherapeutic agent, gemcitabine (Gemzar®; Eli Lilly, IN) that is released from the implant for at least about 2 weeks. Observation of tumor regression is indicative that the treatment is successful. The patient is cured of the pancreatic cancer without requiring surgical removal of the tumor from the pancreas.

### Example 5

### Treatment of Joints with Biodegradable Implants

A 52 year old woman with arthritis is suffering from inflamed joints, especially in her fingers. One implant described in Example 1 which comprises the steroid, dexamethasone, is placed within each joint of the patient's fingers of her left hand. Her right hand is left untreated as a control. The dexamethasone is released for at least about a week. Reduced pain and patient discomfort indicate that the treatment is successful. Reports of reduced pain and discomfort by the patient indicate that the relief provided by the implants persists beyond the time in which the implant releases the dexamethasone.

### Example 6

### Treatment of Stroke with Biodegradable Implants

A 78 year old man who has suffered a stroke receives an intrathecally administered biodegradable implant as set forth in Example 1. The implant comprises brimonidine. Brimonidine is released into the spinal fluid for at least about one week after implantation. Although the patient appears to exhibit sedative side effects, the patient also presents with enhanced brain electrical activity and reduced cellular damage around the stroke area. Successful recovery from the stroke is achieved, and the patient is able to continue to lead his normal living patterns.

### Example 7

### Treatment of Cardiac Conditions with Biodegradable Implants

A 38 year old male suffering from tachycardia is prescribed a biodegradable implant as set forth in Example 1. The implant comprises effective amounts of the beta blocker, propanolol. The implant is placed in proximity to the sinoatrial node. The propanolol is released for extended periods of time and slows the patient's heart beat. After a single administration, a regular heart beat pattern which persists for several months indicates the treatment was successful.

### Example 8

### Treatment of Central Nervous System Cancer with Biodegradable Intraventricular Implants

A 62 year old male suffering from CNS Lymphoma is prescribed a biodegradable intraventricular implant as set forth in Example 1. The implant comprises effective amounts of methotrexate. One implant is placed within each of the patient's ventricles. The methotrexate is released for extended periods of time. Successful treatment of the lymphoma is evidenced by a reduction in tumor size using magnetic resonance imaging (MRI) and/or overall neurologic improvement.

### Example 9

### Treatment of Central Nervous System Cancer with Biodegradable Intraventricular Implants

A 54 year old female diagnosed with ovarian cancer is administered two biodegradable intraperitoneal implants as set forth in Example 1. One implant comprises effective amounts of the anti-neoplastic agent, paclitaxel, and the other implant comprises effective amounts of the anti-neoplastic agent, cisplatin. Both implants are administered intraperitoneally to provide a prolonged combination therapy. The anti-neoplastic agents are released for extended periods of time. Successful treatment of the ovarian cancer is evidenced by a reduction in tumor size using magnetic resonance imaging (MRI).

### Example 10

### Treatment of Dermal Lesions with Biodegradable Dermal Implants

A 48 year old male diagnosed with basal cell carcinoma is administered intradermal implants as set forth in Example 1. The implants comprise effective amounts of the anti-neoplastic agent, 5-fluorouracil, and effective amounts of a photosensitizing agent, such as methyl aminolevulinic acid. Implants are administered intradermally to provide a prolonged combination therapy. The agents are released for extended periods of time, and the patient receives photodynamic therapy to activate the photosensitizing agent. Successful treatment of the carcinoma is evidenced by a reduction in size and/or number of basal cell carcinomas.

All references, articles, patents, applications and publications set forth above are incorporated herein by reference in their entireties.

While this invention has been described with respect to various specific examples and embodiments, it is to be understood that the invention is not limited thereto and that it can be variously practiced within the scope of the following claims.

What is claimed is:

1. A biodegradable non-ophthalmic implant, comprising:
a multi-extruded body member comprising a poly (lactide-co-glycolide) (PLGA) copolymer and a medically useful agent distributed throughout the PLGA copolymer in the form of a non-ophthalmic implant and releasable therefrom to a non-ophthalmic target site of a patient, the PLGA copolymer comprising about 75% by weight acid end PLGA and about 25% by weight ester end PLGA, and comprising about 50% lactide and about 50% glycolide.

2. The implant of claim 1, wherein the body member is a double extruded body member of the PLGA copolymer and the medically useful agent.

3. The implant of claim 1, wherein the medically useful agent is selected from the group of therapeutic agents and diagnostic agents.

4. The implant of claim 1, wherein the medically useful agent is a therapeutic agent homogenously distributed in a PLGA copolymer matrix and is releasable therefrom to provide a therapeutic effect at a non-ophthalmic target site that persists for a time beyond which the therapeutic agent is detectable at the target site.

5. The implant of claim 1, wherein the medically useful agent is selected from the group consisting of chemotherapeutic agents, anti-inflammatory agents, analgesic agents, anti-spasmodic agents, neuroprotective agents, and combinations thereof.

6. The implant of claim 5, wherein the medically useful agent is an anti-inflammatory agent selected from the group consisting of steroids and non-steroidal anti-inflammatory drug.

7. The implant of claim 5, wherein the medically useful agent is an alpha 2 adrenergic receptor agonist.

8. The implant of claim 7, wherein the medically useful agent is selected from the group consisting of brimonidine, salts thereof, and combinations thereof.

9. The implant of claim 1 provided in an implant injecting device.

10. The implant of claim 9, wherein the injecting device comprises a fiber optic system.

11. The implant of claim 1, wherein the body member is a neural implant.

12. The implant of claim 11, wherein the body member is an implant selected from the group consisting of intrathecal implants, intracranial implants, and intraspinal implants.

13. The implant of claim 1, wherein the body member is a cardiac implant.

14. The implant of claim 1, wherein the body member is a joint implant.

15. The implant of claim 1, wherein the body member is selected from the group consisting of pancreatic implants, kidney implants, liver implants, prostate implants, and breast implants.

16. The implant of claim 15, wherein the medically useful agent is a chemotherapeutic agent.

17. The implant of claim 1, wherein the medically useful agent is a therapeutic agent, and the implant releases the therapeutic agent with at least one of reduced side effects, enhanced dosing precision, and reduced frequency of administration relative to systemic administration of an identical therapeutic agent.

18. A method of treating a patient, comprising:
administering a biodegradable non-ophthalmic implant comprising a poly (lactide-co-glycolide) (PLGA) copolymer and a medically useful agent distributed throughout the PLGA copolymer in the form of a non-ophthalmic implant to a non-ophthalmic target site of a patient, the PLGA copolymer comprising about 75% by weight acid end PLGA and about 25% by weight ester end PLGA, and comprising about 50% lactide and about 50% glycolide, wherein the implant releases the medically useful agent to the target site for extended periods of time.

19. The method of claim 18, wherein the implant is administered to a cancerous target site.

20. The method of claim 19, wherein the implant is administered to an organ selected from the group consisting of the brain, the pancreas, the kidneys, the liver, the prostate gland, and the breasts to treat localized cancer tumors or pre-cancerous tissue of the organ.

21. The method of claim 18, wherein the implant is administered to a joint selected from the group consisting of arthritic joints and injured joints to relieve joint pain, joint inflammation, or joint disease.

22. The method of claim 18, wherein the implant is administered to a cardiac target site selected from the group consisting of heart muscle and cardiac arteries to enhance cardiac or vascular function in a patient in need thereof.

23. The method of claim 18, wherein the implant is administered to a target site in the central nervous system of the patient.

24. The method of claim 23, wherein the implant is administered into the spine of the patient, and the implant releases an analgesic or antispasmodic agent to provide long lasting pain or spasticity relief, respectively.

25. The method of claim 18, wherein the medically useful agent is an alpha 2 adrenergic agonist, and the implant is administered intrathecally to provide extended release of low doses of the alpha 2 adrenergic agonist and provide enhanced brain function.

26. The method of claim 18, wherein the implant comprises a steroid and the implant is administered to a central nervous system target site, and the method further comprises administering an implant comprising a chemotherapeutic agent into a different target site to treat a cancerous tumor of the different target site.

27. The method of claim 18, wherein the implant is injected into a target site using an implant injection device.
